(19) United States
(12) Plant Patent Application Publication
(10) Pub. No.: US 2006/0212979 P1
(43) Pub. Date: Sep. 21, 2006

(54) 'Randy' male pistachio

(50) PISTACIA VERA; PISTACHIO

(75) Inventors: Dan E. Parfitt, Davis, CA (US); Joseph Maranto, Bakersfield, CA (US); Craig E. Kallsen, Bakersfield, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 11/085,894

(22) Filed: Mar. 21, 2005

(51) Int. Cl. A01H 5/00

(52) U.S. Cl. PLT/152

(57) ABSTRACT

A new and distinct variety of pistachio tree denominated ‘Randy’ is described. This cultivar is an early flowering male that will be used as a pollenizer for ‘Golden Hills’ and ‘Lost Hills’. It has excellent flowering synchrony with ‘Golden Hills’ and ‘Lost Hills’ and can be used to cover the earlier part of the ‘Kerman’ flowering period during seasons in which ‘Kerman’ flowering is extended. This generally occurs during seasons of low chill, which are expected to become more frequent in the future due to continued global warming. ‘Peters’, the standard male used to pollenize ‘Kerman’, often flowers too late to cover the earlier part of the ‘Kerman’ bloom period under these conditions. ‘Randy’ was selected for high pollen viability, durability, and a high level of pollen production (based on visual evaluation). ‘Randy’ flowers 2 to 4 weeks earlier than ‘Peters’, the standard pollenizer for ‘Kerman’. ‘Peters’ flowers too late to be a very effective pollinator for the new cultivars.

### BOTANICAL/COMMERCIAL CLASSIFICATION

(Pistacia vera)/new Pistachio variety.

### VARIETY DENOMINATION

cv. ‘Randy’.

### BACKGROUND OF THE INVENTION

The present invention relates to a new and distinct variety of Pistachio tree Pistacia vera which has been denominated varietally as ‘Randy,’ and more particularly to such a pistachio tree which has a flowering date of seven to ten days earlier than the industry standard pistachio tree variety ‘Peters’.

‘Randy’ provides good quantities of relatively durable and viable pollen. It has a good flowering overlap period with ‘Golden Hills’, ‘Lost Hills’ and ‘Kerman’ female cultivars and would be used as a pollenizer for these cultivars. ‘Randy’ is more precocious than ‘Peters’, flowering one or two years before ‘Peters’. It would be advantageous to plant ‘Randy’ with Kerman to provide adequate pollination for ‘Kerman’ trees newly coming into bearing and in situations of low chilling when ‘Kerman’ and ‘Peters’ may demonstrate poor synchrony for pollination.

### SUMMARY OF THE INVENTION

It was found that the new cultivar exhibits the following combination of characteristics as compared to ‘Peters’, the industry standard: a) ‘Randy’ flowers 1 to 3 weeks earlier than ‘Peters’; b) ‘Randy’ has significantly more buds per cm branch length and more spur branches than ‘Peters’, suggesting that ‘Randy’ produces many more male inflorescences per tree (and presumably more pollen) than ‘Peters’; c) ‘Randy’ has more durable pollen than ‘Peters’. The pollen stays viable for a longer period, which increases the effective amount of viable pollen available for pollination at any given time; d) Has appropriate flowering period overlap for ‘Golden Hills’ and ‘Lost Hills’. ‘Peters’ flowers too late to serve as a pollenizer for these earlier flowering cultivars; and e) ‘Randy’ also has significantly larger buds than ‘Peters’.

‘Randy’ has been asexually reproduced in Kern County, California and Madera County, California. ‘Randy’ was propagated from buds, inserted into both PGI and UCB-1 rootstocks (budded onto). The cultivar is present at field locations in Kern County, California and Madera County, California (test plots). In addition 2 trees have been budded on UCB-1 rootstocks in pots at Davis for planting into the field this spring.

### BRIEF DESCRIPTION OF THE DRAWINGS

FIG. 1. Comparison photo of ‘Randy’ and ‘Peters’

FIG. 2a. ‘Randy’ Inflorescences on Tree—Mar. 31, 2004

FIG. 2b. ‘Randy’ Inflorescences on Tree—Mar. 31, 2004

FIG. 3a. ‘Peters’ Inflorescences on Tree—Mar. 31, 2004.

FIG. 3b. ‘Peters’ Inflorescences on Tree—Mar. 31, 2004.

FIG. 4. ‘Randy’ tree taken on Apr. 11, 2002 at the Kern Co. test plot near Lost Hills.

FIG. 5. ‘Randy’ at the Kern Co. advanced selection test plot, on Mar. 27, 2003.

FIG. 6. Standardized comparison of ‘Randy’ and ‘Peters’ inflorescence and new leaves—Mar. 31, 2004.

FIG. 7. Two ‘Peters’ trees flanking ‘Randy’ tree in center. Note larger tree size and large number of spur branches for ‘Randy’.

FIG. 8A. ‘Randy’ branch showing spurs.

FIG. 8B. ‘Peters’ branch showing lack of spur branching.

### DETAILED BOTANICAL DESCRIPTION

The following description describes the key characteristics of the new male pistachio cultivar named ‘Randy’ as well as reference to the standard pistachio cultivar ‘Peters’ used as a pollenizer in California.
The cross: The cross that produced ‘Randy’ was made in 1990, and the progeny planted in 1991. The cross is between a Pistacia vera female ‘2-35’, located in Kern County and propagated from buds supplied to Joseph Maranto from a plot in UC Davis in 1985, and a Pistacia vera male ‘ES#3’ originally from the Plant Introduction Garden at Chico Calif. ES#3 is no longer available. This seedling was designated as B15-31. In 1997, a replicated yield trial was established near Lost Hills Calif., on a Paramount Farming Company ranch. In this plot, ‘Randy’ was budded onto either UCB-1 or PG-1 rootstock planted at the ends of alternate rows (alternating with ‘Peters’ male) between the female replicates. ‘Randy’ first flowered in 2000. A second replicated yield trial was established north of Fresno on an S&J Ranch farm in September 1999. In this plot, ‘Randy’ is located at the ends of the rows, in alternate rows with ‘Peters’ and grafted to UCB-1 or PG-1 rootstocks. In this plot ‘Randy’ flowered in 2003.

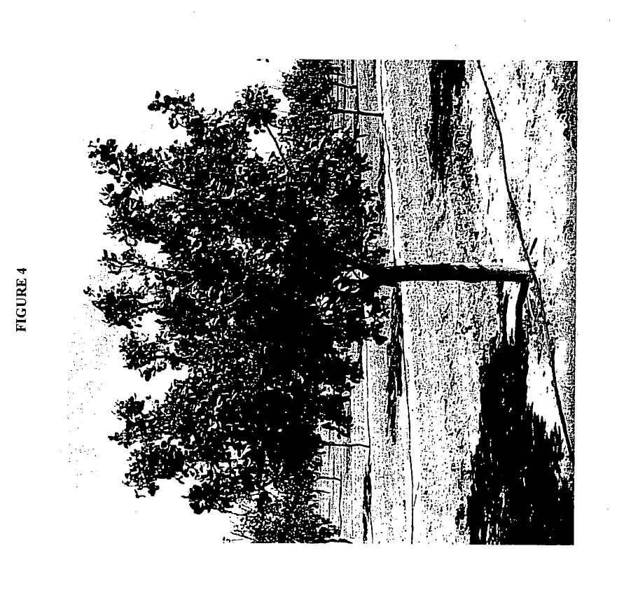

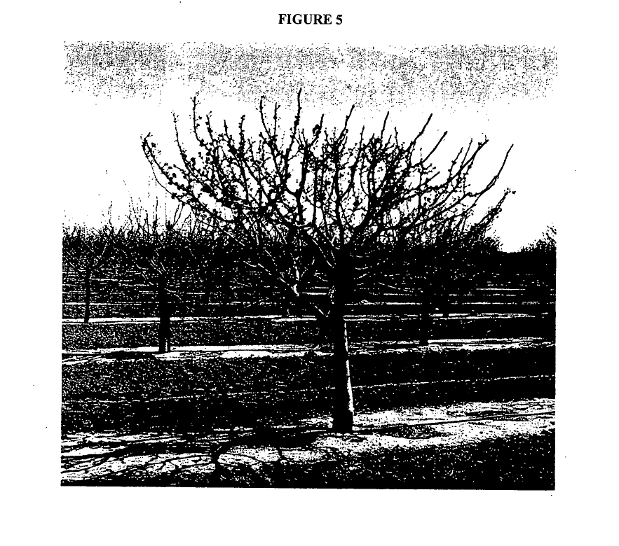

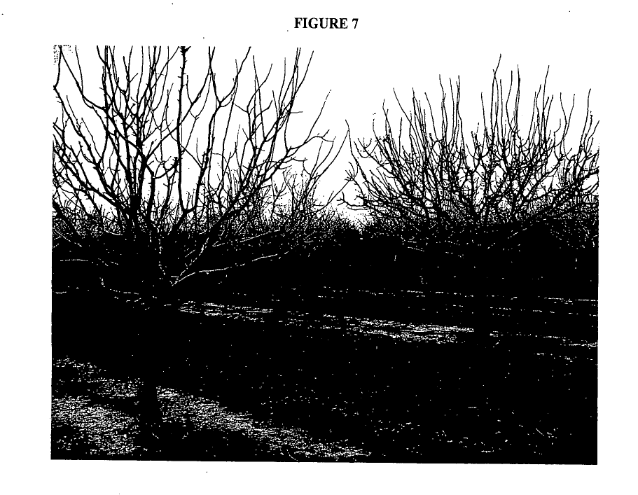

Tree vigor: The tree is of average size for a pistachio, based on observation of 7 year old trees. Grafted trees are about 3 m tall at 7 years with a spread equal to the height. Trunk diameters are 10 to 15 cm. It is expected that ‘Randy’ will eventually be a larger tree than the female ‘Kerman’. See FIGS. 4-5. ‘Randy’ is a significantly larger tree based on measured cross sectional area than ‘Peters’, although there may be some confounding rootstock effect for this character (ANOVA below). Close up photo evaluation of trunk lenticels was undertaken. However, no obvious or significant differences in pattern, number, or appearance were seen between ‘Randy’ and ‘Peters’. See Tables 1 and 2 and FIG. 7.

TABLE 1
ANOVA Table for xsec. area

| | DF | Sum of Squares | Mean Square |
|---|---|---|---|
| Cultivar | 1 | 8413324.610 | 8413324.610 |
| Residual | 8 | 5476071.158 | 684508.895 |

| | F-Value | P-Value | Lambda | Power |
|---|---|---|---|---|
| Cultivar | 12.291 | .0080 | 12.291 | .876 |

Means Tables for xsec. area
Effect: Cultivar

| | Count | Mean | Std. Dev. | Std. Err. |
|---|---|---|---|---|
| Peters | 5 | 3239.631 | 299.697 | 134.028 |
| Randy | 5 | 5074.114 | 1141.017 | 505.806 |

TABLE 2
Fisher's PLSD for xsec. area
Effect: Cultivar
Significance Level: 5%

| | Mean Diff. | Crit. Diff. | P-Value | |
|---|---|---|---|---|
| Peters, Randy | −1834.484 | 1206.645 | .0080 | S |

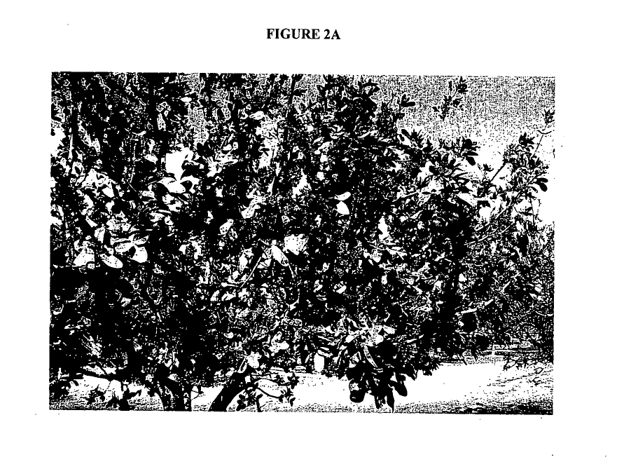

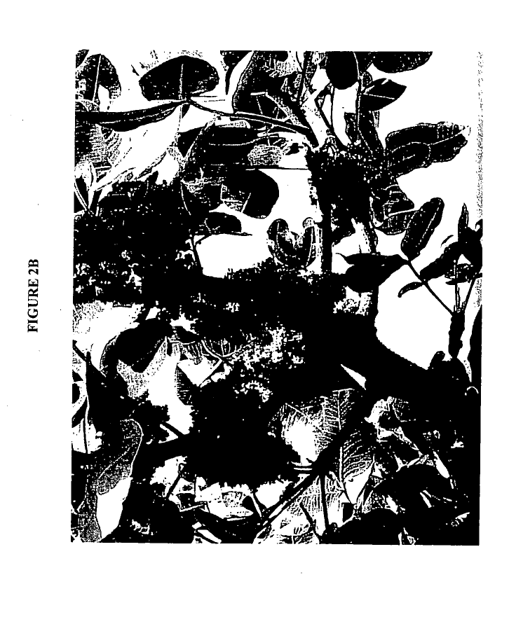

Tree structure: ‘Randy’ has tree structure and branching habit typical for Pistacia vera L. (FIGS. 4 and 5). Branch angles are broad, ranging from 80 to 90 degrees for both scaffold and lateral branches (FIG. 2b). Distribution of scaffold and lateral branches is a function of pruning and training activities which are practiced intensively during the first three years of growth.
Flower buds: ‘Randy’ has significantly more buds per cm branch length than ‘Peters’.

10 branches were measured on each of 5 trees of ‘Randy’ and ‘Peters’. ANOVAs were run as a completely random design (observations not grouped by tree). See Tables 3 and 4.

TABLE 3
ANOVA Table for buds/cm

| | | Sum of | Mean | | | | |
|---|---|---|---|---|---|---|---|
| | DF | Squares | Square | F-Value | P-Value | Lambda | Power |
| Cultivar | 1 | 1.973 | 1.973 | 109.442 | <.0001 | 109.442 | 1.000 |
| Residual | 98 | 1.767 | .018 | | | | |

Means Table for buds/cm
Effect: Cultivar

| | Count | Mean | Std. Dev. | Std. Err. |
|---|---|---|---|---|
| Peters | 50 | .187 | .124 | .018 |
| Randy | 50 | .468 | .144 | .020 |

TABLE 4
Fisher's PLSD for buds/cm
Effect: Cultivar
Significance Level: 5%

| | Mean Diff. | Crit. Diff. | P-Value | |
|---|---|---|---|---|
| Peters, Randy | −.281 | .053 | <.0001 | S |

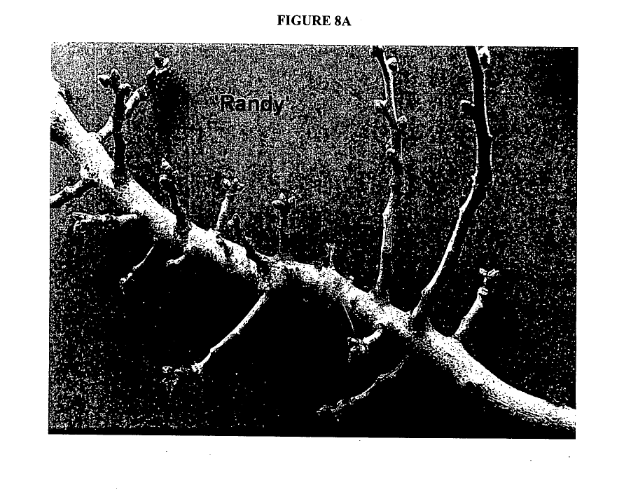

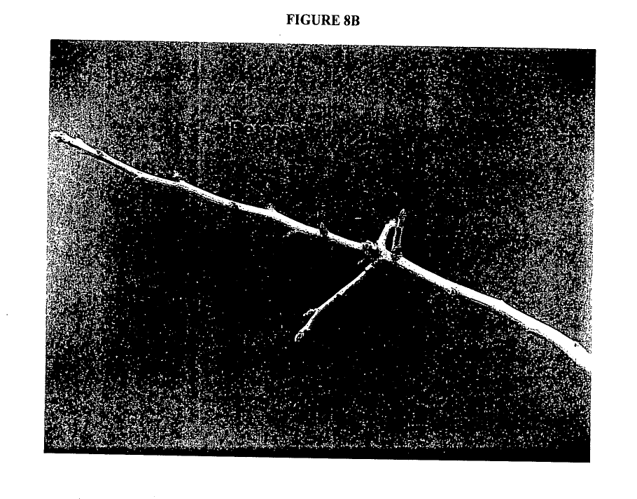

‘Randy’ had more than twice as many buds per cm of flowering branch as ‘Peters’. While this was not quantified, it was also observed that Randy has more spur branches than ‘Peters’. See FIGS. 8A and 8B. This effect is quite pronounced, and diagnostic. The larger number of spur branches and buds/cm of branch means that ‘Randy’ produces many more male inflorescences per tree (and presumably more pollen) than ‘Peters.’

‘Randy’ also has significantly larger buds than ‘Peters’. 10 buds per tree were measured for width by length in mm. Length values for both sets of data were similar and non-significantly different, but ‘Randy’ buds were about 20% wider than ‘Peters’ buds, resulting in highly significant volume differences. See Tables 5 and 6.

TABLE 5

| | | | | | | |
|---|---|---|---|---|---|---|
| | Sum of | Mean | | | | |
| DF | Squares | Square | F-Value | P-Value | Lambda | Power |

ANOVA Table for bud vol (mm\hat{ }3)

| | | | | | | | |
|---|---|---|---|---|---|---|---|
| Cultivar | 1 | 171.185 | 171.185 | 57.565 | <.0001 | 57.565 | 1.000 |
| Residual | 98 | 291.427 | 2.974 | | | | |

ANOVA Table for bud l (mm)

| | | | | | | | |
|---|---|---|---|---|---|---|---|
| Cultivar | 1 | .640 | .640 | .708 | .4023 | .708 | .127 |
| Residual | 98 | 88.626 | .904 | | | | |

ANOVA Table for bud w

| | | | | | | | |
|---|---|---|---|---|---|---|---|
| Cultivar | 1 | 139.240 | 139.240 | 79.116 | <.0001 | 79.116 | 1.000 |
| Residual | 98 | 172.476 | 1.760 | | | | |

TABLE 6

| | | | |
|---|---|---|---|
| Count | Mean | Std. Dev. | Std. Err. |

Means Table for bud vol (mm{circumflex over ( 3)
Effect: Cultivar

| | | | | |
|---|---|---|---|---|
| Peters | 50 | 4.226 | 1.356 | .192 |
| Randy | 50 | 6.843 | 2.027 | .287 |

Means Table for bud l (mm)
Effect: Cultivar

| | | | | |
|---|---|---|---|---|
| Peters | 50 | 11.424 | .941 | .133 |
| Randy | 50 | 11.264 | .961 | .136 |

Means Table for bud w
Effect: Cultivar

| | | | | |
|---|---|---|---|---|
| Peters | 50 | 8.482 | 1.256 | .178 |
| Randy | 50 | 10.842 | 1.393 | .197 |

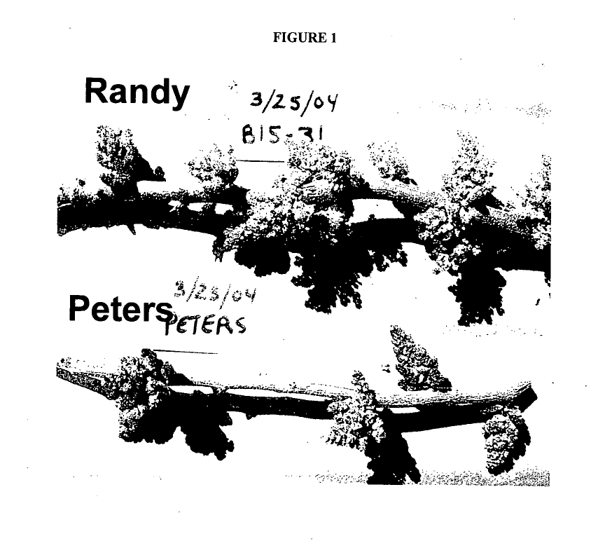

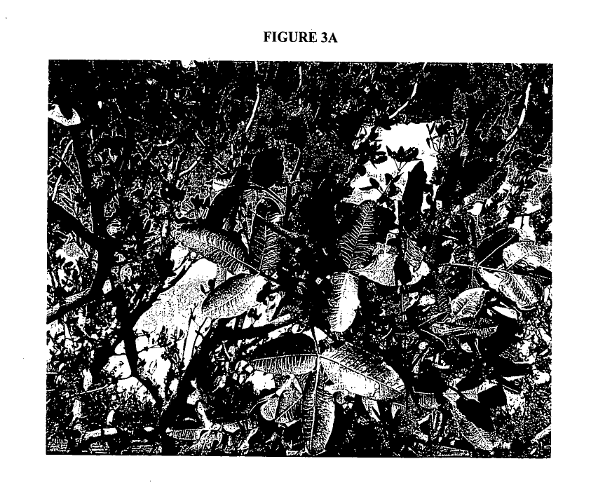

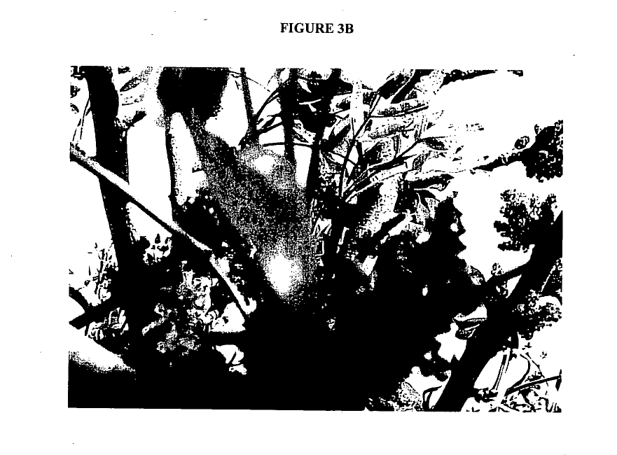

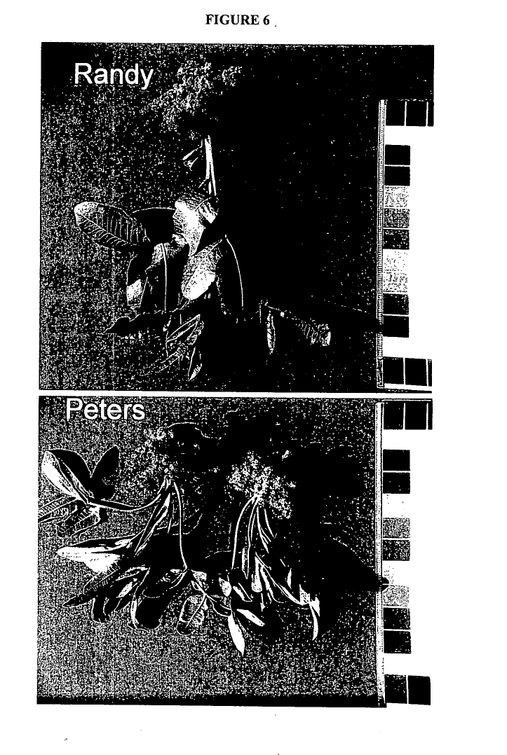

Inflorescences: Female inflorescences are born laterally alternately on branches, rarely as terminal buds. They are located on one year old wood. The flower buds form a branched compound inflorescence of the panicle form. Individual flowers are 0.5 mm to 1 mm in size. All flowers are male. The panicles are 3 to 7 cm long with considerable variation in size. Flower development is from base to tip of the panicle and typically spans a 3 week period, depending on weather conditions during individual seasons. Panicles are yellow as is the pollen. Tips of the panicles are tinged red prior to opening of individual flowers. FIGS. 1 and 6 show a comparison of ‘Randy’ vs. ‘Peters’ inflorescences. FIGS. 2a and b show ‘Randy’ inflorescences on the tree and FIGS. 3a and b show ‘Peters’ inflorescences on the same date (Mar. 31, 2004). FIG. 6 shows a standardized comparison of ‘Randy’ and ‘Peters’ inflorescence and new leaves.
Pollen viability: Pollen viability was measured with hanging drop slides in 1996. ‘Randy’ has highly viable and durable pollen. Initial readings (Apr. 30, 1996) were 75% viable declining to 35% viable after 29 days of storage. ‘Peters’ initial viability was 45%, declining to 5% to 15% after 24 to 27 days. The pollen produced by appears to be more durable than that produced by ‘Peters’.
Flowering date: ‘Randy’ flowers 1 to 3 weeks earlier than ‘Peters’. It is characterized by a relatively long bloom period, in excess of 2 weeks, a characteristic that ‘Peters’ shares. Most male pistachios have a flowering period of about one week. Peak flowering is a week earlier than for ‘Kerman’, approximately 2-3 days earlier than for ‘Kerman’ when sprayed with Volck supreme oil (470 wt) in 2003 and approximately one week earlier than ‘Peters’ depending on chilling hours. (Oil sprays are used to advance flowering by the industry and tend to compress the bloom period).

Data from seedling test plot in Kern Co.:

1995: For ‘Randy’—peak flowering Mar. 18, 1995 to Mar. 24, 1995

1996: For ‘Randy’—Apr. 3, 1996 to Apr. 10, 1996; for ‘Peters’—Apr. 23, 1996 to May 3, 1996

1997: For ‘Randy’—first flowering Mar. 24, 1997, peak flowering Apr. 1, 1997 to Apr. 4, 1997, last flowering Apr. 7, 1997 to Apr. 10, 1997

1998: For ‘Randy’—first flowering Mar. 30, 1998-Apr. 5, 1998, peak flowering Apr. 6, 1998-Apr. 12, 1998, last flowering Apr. 13, 1998 to Apr. 20, 1998

1999: For ‘Randy’—first flowering Mar. 8, 1999 to Mar. 12, 1999, peak flowering Mar. 15, 1999 to Mar. 19, 1999, last flowering Mar. 22, 1999 to Mar. 26, 1999

2000: For ‘Randy’—first flowering Apr. 3, 2000 to Apr. 7, 2000

Data from grafted test plot in Kern Co. Trees were grafted on either ‘UCB1’ or ‘Pioneer Gold 1’ rootstocks. Visits to the two experimental sites were made at intervals of three to four days through the bloom period. In 2004 (8^{th} year since grafting), a bloom-rating of 1 through 6 was used with 1=dormant; 2=early bloom, 3=mid bloom, 4=full bloom and 5=late bloom. Bloom evaluation is subjective; the number of individual flowers in bloom within an inflorescence varies, as does the degree of flowering at different locations along a branch. Full bloom was an estimate of when the maximum number of receptive stigmas was present on the tree. On Mar. 25, 2004 ‘Randy’ was past full bloom (4.5), ‘Peters’ was at early bloom (2.5).
Quantity of inflorescences: A visual score (low, medium, and high) was used to evaluate the 656 male seedlings at the Kern Co. seedling plot in 1997 and 1998. ‘Randy’ was scored as having a high number of inflorescences.
Leaves: Leaves are simple compound imparapinnate with 1 to 2 pairs of oppositely arranged leaflets. Leaves are 10 cm to 15 cm long with 5 cm to 8cm leaflets. Leaves vary considerably in shape, in general being ovate with cuspidate to rounded tips and rounded base. Margins of leaf blades are entire. Leaf surfaces are glabrous, smooth and waxy. Leaves range from light green at first emergence to dark green at maturity FIGS. 2a, 2b.
Leafing date:

1997: For ‘Randy’—first leafing 4/7/97 to 4/10/97

1998: For ‘Randy’—first leafing 4/13/98 to 4/20/98

1999: For ‘Randy’—first leafing 3/15/99 to 3/19/99

2000: For ‘Randy’—first leafing 4/3/00 to 4/7/00

‘Randy’ flowers earlier than ‘Kerman’ and, like ‘Peters’ has durable pollen. ‘Randy’ will be a superior pollinizer for the new female varieties ‘Golden Hills’ and ‘Lost Hills’ or any other variety which flowers up to 10 days earlier than Kerman. ‘Randy’ males would be useful in existing ‘Kerman’ orchards during seasons of low chilling or for other conditions when the flowering synchrony is poor, as ‘Peters’ is the tree that most often demonstrates flowering delay. ‘Randy’ grows quickly and is one to two years more precocious than ‘Peters’, which is a significant advantage in new orchards just coming into bearing

What we claim is:

1. A new and distinct variety of pistachio tree substantially as shown and described herein.
